(19) United States
(12) Patent Application Publication
(10) Pub. No.: US 2013/0096598 A1
(43) Pub. Date: Apr. 18, 2013

(54) UNIVERSAL CLOSURE AND METHOD OF LUBRICATION

(71) Applicant: Erblan Surgical, Inc., Temecula, CA (US)

(72) Inventor: Ernesto E. BLANCO, Temecula, CA (US)

(73) Assignee: Erblan Surgical, Inc., Temecula, CA (US)

(21) Appl. No.: 13/705,598

(22) Filed: Dec. 5, 2012

Related U.S. Application Data
(62) Division of application No. 12/607,593, filed on Oct. 28, 2009.
(60) Provisional application No. 61/110,783, filed on Nov. 3, 2008.

(51) Int. Cl. A61B 17/34

(52) U.S. Cl. CPC A61B 17/34
USPC 606/185

(57) ABSTRACT

A seal assembly includes a first seal and a second seal and positioned in the first end element. The second seal can include at least one longitudinal groove therein and have members connected thereto. A stopper member is coaxially positionable within the second seal and the first seal and a lubricant can be positioned between the stopper member and the second seal. The seal assembly can also be integrated with a capsule for removable insertion within a tubular member.

### CROSS REFERENCE TO RELATED APPLICATION

This application is a Divisional of and is based upon and claims the benefit of priority under 35 U.S.C. §120 for U.S. application Ser. No. 12/607,593, filed Oct. 28, 2009 which claims the benefit of U.S. Ser. No. 61/110,783, filed Nov. 3, 2008, the entire contents and disclosure of each of which are herein incorporated by reference.

### BACKGROUND OF THE INVENTION

### Field of the Invention

The present application comprises a universal closure that provides for a reduction in the number of parts necessary to form an introducer assembly of the type shown in the prior application and permits the utilization of a single seal in combination with a closing valve. The seal optionally includes a unitary seal assembly which utilizes a plurality of relief grooves on the seal. Both the closing valve and unitary seal are also provided with a chamfered front edge portion, with the seal being optionally provided with the plurality of grooves in order to assist in flexibility of the seal and to assist in the insertion therethrough of an obturator or other medical device during surgery, for example, and the subsequent withdrawal of the same there through. A preferred embodiment of the same includes relief grooves provided solely on the exterior of the seal.

Applicant has also recognized the advantage of providing for lubrication of the seal and has therefore conceived of the ability of providing a lubricant contained within a portion of the closure assembly, the lubricant being maintained in place by a removable lid having a projection extending therefrom which extends towards a tapered end portion of the seal, as explained below. Accordingly, the lubricant can be stored between the removable lid and the distal portion of the projection so as to permit removal of the lid immediately prior to insertion of the cannula or other medical device through the seal.

For example, the seal can be utilized in any structural assembly that permits passage of a member through a seal. This could include, for example, the nozzle of a filling station pump that passes through a seal mounted in the passageway of the vehicle that leads to the fuel tank of the vehicle. Other possibilities are clearly possible which would be within the knowledge of one of ordinary skill in the art of providing seals for passageways. It is therefore to be understood that within the scope of the pending claims, the invention may be practiced otherwise than as specifically described therein.

### SUMMARY OF THE INVENTION

The present invention is directed to a complex universal system in which all the elements comprising the same title are formed as one single unit.

Simplicity and compactness are desirable in cannula closures along with high reliability. The system involved here is not just desirable by surgeons but also by manufacturers because its design elements are simpler in structure and are easier to fabricate and assemble.

The most common complaint of endoscopic surgeons is the loss of insufflation gas due to leakage across cannula closures or seals of a trocar. Such leakage may be caused by the design complexity of the instruments which may be difficult to introduce or manipulate during surgical procedures, thereby causing gas leakage and seal ruptures. These complaints have required extreme attention from suppliers but unfortunately have resulted in higher closure complexity and cost while in some cases compounding surgical dissatisfaction and accidents.

The design covered in the present invention comprises a geometrical combination of critical elements within a single unitary seal-valve unit. The seal includes a rather hard durometer, conical element, the orifice of which is made so as to have stiffness in the axial direction to control snagging deformations, and which is axially grooved externally to minimize hoop stresses which would cause circumferential stiffness and hard entry forces. The result is a cone that could be described as being formed by a combination of axial beams spaced by hoop-relief grooves that allow it to expand as a radially soft-hoop element while exposing a harder and low friction internal surface to contact with incoming sharp penetrating instruments. The results is a best-of-all-worlds design which does not incur the complexity of present kinematic opening systems prone to break and to either cause closure failures, or risk the release of detritus within the surgical field.

In addition to those characteristics of the novel conical orifice of the present invention is the fact that it is cast within an external conoid that completes the whole elastic closure system by ending in a bivalve, or linear slit valve, which is sufficiently stretched along the slit opening so as to insure an absolutely reliable, not just static, but forcible closure, when instruments are withdrawn.

Since this design is cast in one piece it also offers the characteristic of not allowing reversibility across the conical support base at the joint between the two cones. Such joint between the external conical-flat seal (a conoid surface) and the internal orifice cone, also known as an aperture, is not a circular joint as would appear at first sight but rather has a spatial perimeter that extends radially and axially between the two surfaces. As a result, the central orifice is supported close to the distal opening portion thereof at two sides, and at right angles it is attached to the conoid further proximally to a base thereof. The result of such configuration is to further increase the integrity of the inside orifice cone while allowing greater radial elasticity at two of its sides.

Since the two elements comprising the elastic seal are desired to have great radial elasticity, it is also logical to minimize the radial constraint imposed by the outside conoid. Therefore a set of longitudinal relief grooves is molded on the outside surface of the conoid surrounding the orifice cone. As a result, a hard durometer, low friction, seal aperture is obtained of great radial elasticity and with greater surface strength than is otherwise possible.

All of these are very desirable factors. Moreover, in the present design, an additional and very important improvement was added. Since the ease of penetration is such a critical requirement, it was decided to introduce a lubrication system into the closure described above. To do so it was necessary to guarantee that fresh fluid lubrication is available for the closure at the start of penetration and without compromising clean room essential procedures and demands on personnel.

As shown in this invention, all of those considerations have been met to full satisfaction. What was done was to use a viscous lubricant of biocompatible characteristics, such as a hydrogel like hyaluronic acid (or hyaluronan) or a comparable substance component of the human body. Hyaluronic acid is the only non-sulphated glycosaminoglycan that is found throughout the body in tissues and fluids. It is an excellent lubricant for limited periods of usage and therefore most adequate for the needs contemplated here. It is only necessary to maintain it in an enclosure to insure its use at any reasonable time after packaging.

The present invention provides the suggestion of this particular hydrogel. However, other suitable biocompatible lubricants can also be employed in this particular application. What is critical in the case of the universal cannula closure involved here is the manner in which a lubricant must be contained within a space in the closure and how it should be delivered prior to its use with the cannula.

First of all the closure should be packaged inside the cannula and not be opened until the start of a procedure. In the case of a cannula for use with a trocar the insertion of the trocar, this should be done at the time of usage. The cannula and the trocar could be packaged in separate blisters of the same package and used as indicated.

The viscous lubricant must be completely sealed within the inside of the cone at the inlet of the seal. Such a “sealed-in” space is obtained by a plugging device that will dilate and plug the orifice at the distal end of the cone and allow the lubricant to partly fill the inside portion thereof around the plug within the cone, while having a plastic cover at the proximal end which will be soft contact-bonded to the outer edges of the seal. Such a plug-and-bonded closing cover can be simply peeled off at surgery time exposing the open freshly lubricated entry space for the surgical instruments to slide in easily without forcibly pushing them.

The described seal lubricated closure becomes, in essence, a double ended closed bottle of lubricant freshly available to be doubly opened at the time of surgery. Such a design and method could be used in other areas where a double-sealed substance must be freshly delivered without encumberant risks to surgical assistants.

While the peeled-off outside of the described enclosure may not be fully protectable, the fact that the proposed system performs two simultaneous double-sealing and double opening tasks appears to be new in this field.

In addition to the described parts of this lubricated closure, the plug sealing the distal end may be hollow and be partially filled with lubricant, which in turn may be delivered through a set of wall holes when the cover center is depressed inwardly, therefore becoming a lubricant reserve if the cover is left attached at one side of the seal after partial peeling. Such an approach would be advantageous for longer term procedures requiring the passing of many instruments.

Conceivably, lubricant-filled seals of different types could be identified by color or symbols.

### DESCRIPTION OF THE DRAWINGS

FIG. 1 is a top, front and right side perspective view of the universal closure;

FIG. 2 is an enlarged view of an end portion of the internal cone shown in FIG. 1;

FIG. 3 is a cross-sectional view of the universal closure shown in FIG. 1;

FIG. 4 is a rear elevational view of the universal closure shown in FIG. 1;

FIG. 5 is a front elevational view of a casting assembly utilizing the universal closure shown in FIG. 1;

FIG. 6 is an exploded side view thereof showing a closing plug and housing;

FIG. 7 is a top and side perspective view of a casting assembly when assembled;

FIG. 8 is a cross-sectional view thereof;

FIG. 9 is a cross-sectional view showing the casting assembly upon removal of the closing plug;

FIG. 10 is a cross-sectional view showing an additional embodiment which includes a disk stopper and lubricant;

FIG. 11 illustrates the embodiment of FIG. 10 with the disk stopper removed;

FIG. 12 is a bottom, front and left side perspective view of a third embodiment of the invention;

FIG. 13 is a cross-sectional view thereof;

FIG. 14 is a front elevational view thereof;

FIG. 15 is a side cross-sectional view thereof; and

FIG. 16 is a bottom plan view thereof, the top plan view being a mirror image of the bottom plan view shown.

### DETAILED DESCRIPTION OF THE PREFERRED EMBODIMENTS

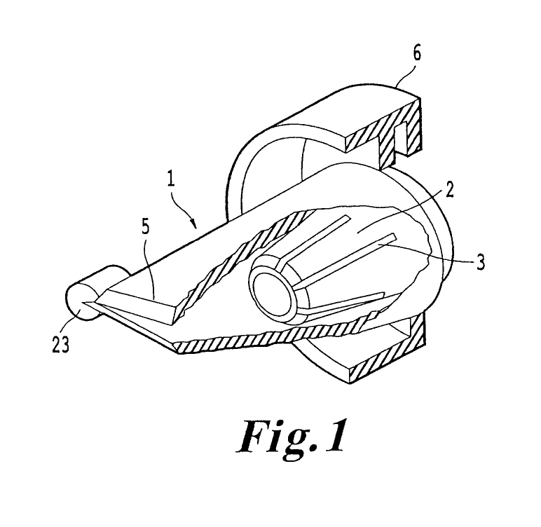

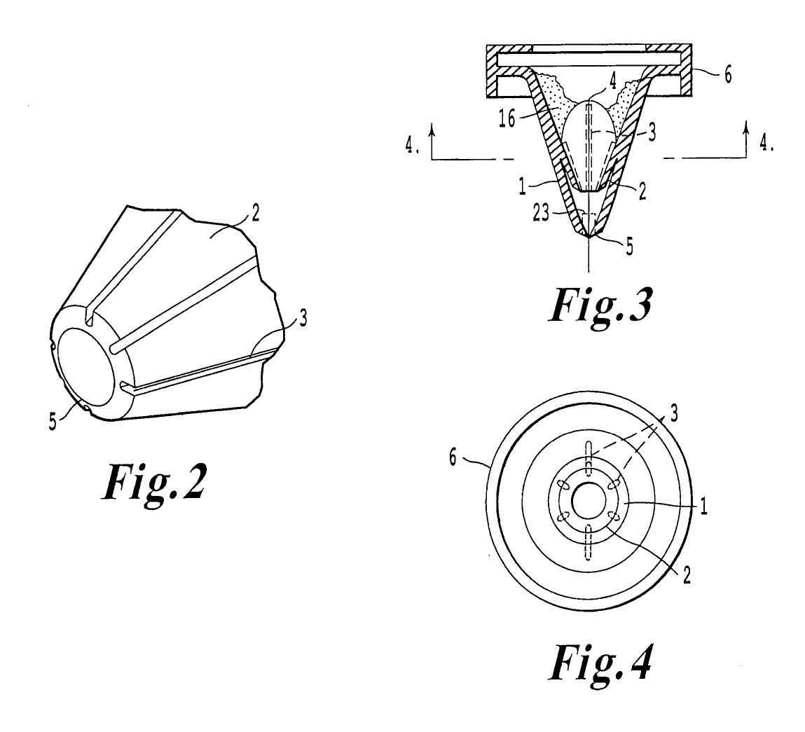

With reference to FIGS. 1-4, a conoid 1 containing an internal cone 2 as an integral part and is also formed with an attachment rim 6 to fit a cannula (not shown). FIG. 4 is a sectional view which also shows the perimeter joining the two conical elements as a line 4, shown in dotted lines. A chamfer 5 of a 45° angle is provided on the distal end of each of the conoid 1 and cone 2. Cone 2 is cast jointly with conoid 1 and to form a one piece assembly which includes at the outer surface thereof six longitudinal relief grooves 3 for reducing hoop stress and to facilitate dilation for entering surgical instruments and reduce insertion forces. The conoid 1 forms a closing hole for the universal closure. The cone 2 can function, moreover, without such relief grooves 3, if desired. As a result, cone 2 comprises a strong, axially firm cone, with a radial opening softness characteristic without snagging surgical instruments inserted there through or withdrawn therefrom. These new design features make it unnecessary to resort to additional opening mechanical means to facilitate orifice dilation. In other words, cone 2 is an example of the use of stress analysis to simplify design by controlling strains in the material of the conoid 1 and cone 2 in order to obtain a desired behavior, without the need for additional kinematic complexity, as is often otherwise required. This can be accomplished by applying relief grooves 3, 9 to the exterior of the outer conoid surface 1 surrounding the inside cone 2, as shown in FIG. 1-4, if desired. Conoid 1 has an elongated opening as shown in FIG. 1 while the adjacent opening of cone 2 is substantial circular in shape.

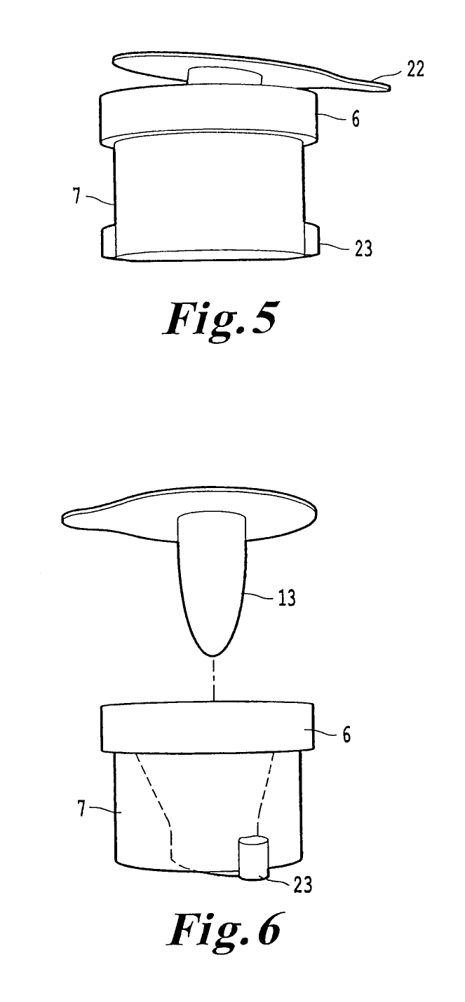

In FIGS. 5-9, the conical integration casting 1, 2 is shown positioned in a cylinder 7 that serves as a seal housing as shown in the left side cross section shown in FIG. 9. In FIG. 9, the relief grooves 9 are shown on the exterior of conoid 1 directly outside of cone 2. The purpose of relief grooves 9 on the conoid 1 is exactly the same as the grooves 3 on cone 2. Such grooves are intended as a novel means to facilitate radial expansion without compromising overall functional integrity. Such is a little known, but useful, novel design approach for strength and simplicity in the present invention.

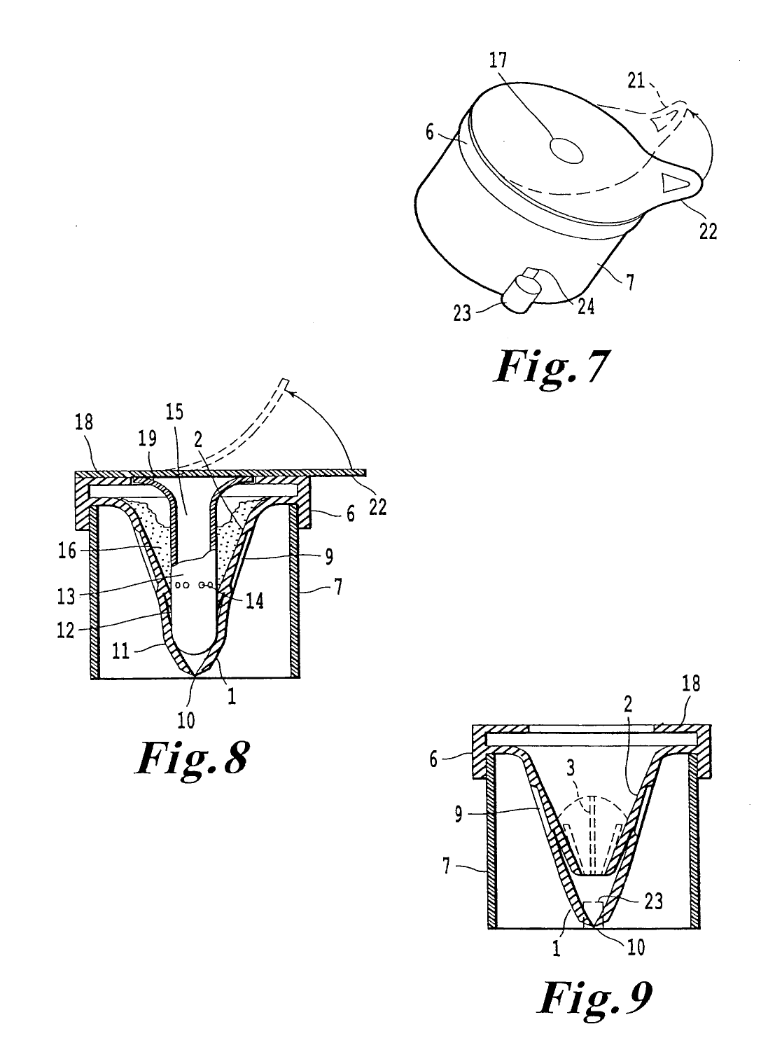

In the design shown in FIGS. 7-9, the line-vertex of the conoid 1 is shown as ending at the juncture of the two sheets of the surfaces thereof at vertex 10. The juncture at vertex 10 between the two beveled lips of the conoid 1 have at each opposite ends a locking knob 23 insertable into a slot 24 formed in each side of the housing 7 to force a substantial stretching of the lips of 1 against each other and to assure a tight closing at all times, except when opened by a penetrating surgical instrument such as an obturator of a trocar upon insertion of the seal within the curvature of the trocar. The higher gas pressure upon insufflation in the patient must never be assumed to guarantee proper lip sealing. FIGS. 7-9 depict graphically the intended lubricant containment within the interior space of this seal.

As is well known, any water-based fluid must be maintained within air-tight containers. In the case of the self lubricated seal of the present invention, the lubricant 16 is contained within the center spaces inside the seal 2 defined between an inserted closing plug 13 between a point 11 at the distal end thereof and a cap 18, attached to the proximal base 19 of the plug. Therefore, all the spaces between the dilated orifices at point 11, seal 12 and the proximal cap 18 containing lubricant 16 are completely air-tight. At the moment of surgical need, the tab 22 can be stripped from position 22 to position 21 and thus be opened for insertion of surgical instruments. The tab 22 then can be either discarded or left partly attached for further use, if necessary. In the latter case additional lubricant can be ejected by reinserting the plug 13 and the pressing region 17. Such action will release from the plug 13 some additional lubricant 16 from the space 15 through a series of openings 14 around the surface of the plug.

It is noted that a double-opening container of the described type may have extensive uses in medical applications for clinical examinations as well as surgical uses.

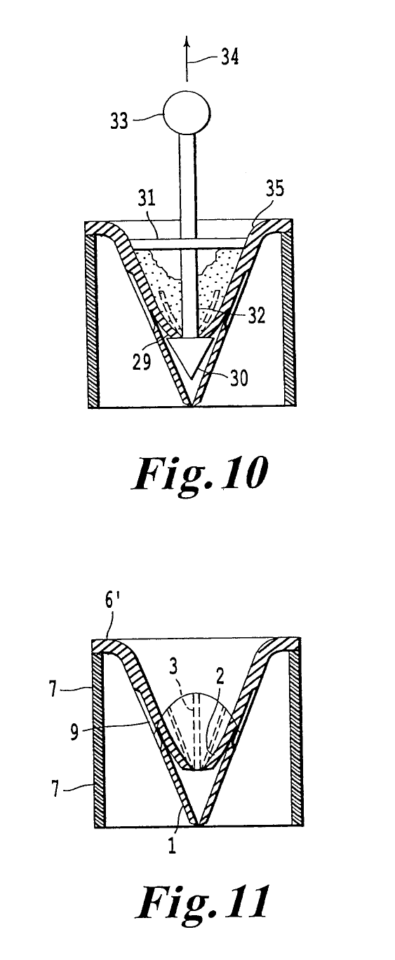

FIGS. 10 and 11 describe an additional preferred embodiment for fluid lubricant containment within the space inside the seal cone and illustrate a double stopper lubricated closure. This design has even better handing and effectiveness than the embodiment shown in FIGS. 5-9.

The design in FIGS. 10 and 11 has only one double-stopper element which provides very positive sealing, assembly, and removal characteristics. The very tight fit between the seal cone inside surfaces obtained by axial compression between the small cone orifice and the slanted periphery of the flat disk 31 shown assures an air-tight internal containment for the lubricant for an indefinite period of time. In addition to that, wetting of the contacting surfaces of the stoppers 30 and 31 at assembly further improves joint sealing since the externally air-exposed edges dry out softly onto each other in a fluid-molding manner which resist shaking and thermal effects without affecting the lubrication performance.

The cost of the parts and the assembly thereof are also reduced since there is no need to bond a cover 22 to the proximal surface, and only a firm pull on handle member 33 will snap off the double-stopper while the lubricant will be partly moved toward the inside by the elastic recovery of the cone. In other words, a number of improvements serve to potentially favor this design over that shown in FIGS. 5-9.

The proposed double-stopper container shown in FIGS. 10 and 11 include a single molded part comprising four elements including the cone 30, element 31, a stem 32 and a knob 33, for being firmly inserted across the cone 2, until the cone point 30 snaps beyond the silicone orifice and is then released, causing the traversed cone 2 to be compressed axially between the proximal flat surface of cone point 30 and the distal surface of element 31. As a result, the narrower cone internal surfaces of the cone 2 will be pushed radially inwardly as shown by member 29 and become tightened over the end surfaces of the stem 32 while the disk stopper 31 pushes radially outwardly and distally against the proximal region of the silicone seal cone at location 35. Such simultaneous elastic deformation results in a sealing effect for the lubricating fluid deposited into cone 2 insuring a truly air-tight space for the lubricant.

The design in FIGS. 10 and 11 also depicts a simple external region 6′ as compared with the one shown in FIGS. 5-9. Region 6′ can be bonded at side portion 28 onto the rim of housing 7, therefore rendering the whole seal suitable for face mounting axially against a cannula internal rim at region 6′.

The advantages of the universal closure for the present filed invention are that a single seal can be provided so as to reduce the number of parts necessary to form the universal seal, while maintaining sufficient flexibility and providing a tight seal around the obturator or other surgical equipment passed through the seal. Such seal also has the advantage of being a one-piece element. More particularly, a single casting molded silicone piece is preferable. The utilization of the lubricant also has the distinct and novel advantage of assisting in entry of the obturator and withdrawal of the same. Such lubricant can be a biological substance having lubrication properties such as a hydrogel such as a hyaluronic acid so as to provide a preferred smooth and reliable lubrication not presently available in conventional seals. Providing the lubricant beneath the removable lid so as to be housed between the lid, the seal and an end portion of the projection permits the lubricant to be securely housed within the seal and to maintain its lubrication properties.

An additional advantage provided by the seal shown in FIGS. 1-9 is that force reduction can be obtained by the utilization of thinner walls in the seal reinforced by longitudinal thicknesses between the grooves so as to prevent snagging and ripping of the seal opening. Such thinner walls can be made, for example, from a higher durometer (40-50, for example) material to obtain strength and a low friction coefficient while maintaining the tightness over the instrument so as to be relatively low. Such therefore requires the utilization of a higher strength durometer characteristic of the seal. A similar performance could potentially be obtained through the use of a lower strength durometer (i.e., 20-30, for example) and in making the seal walls thicker, however, such design inevitably would entail a higher friction coefficient which could be detrimental to penetration forces and enhanced snagging by sharp instruments with the walls of the seal and valve. Such snagging is a major problem and is even more of a problem than penetration force difficulties since it induces ripping which destroys the orifice of the seal if not properly designed. However, the present invention serves to prevent this type of problem.

In summary, a lower wall resistance as a result of friction can be obtained without the danger of snagging through harder wall surfaces which have a lower friction coefficient while still maintaining the strength needed by the utilization of longitudinal corrugations provided along the outside surface of the seal cone, terminating just short of the orifice thereof.

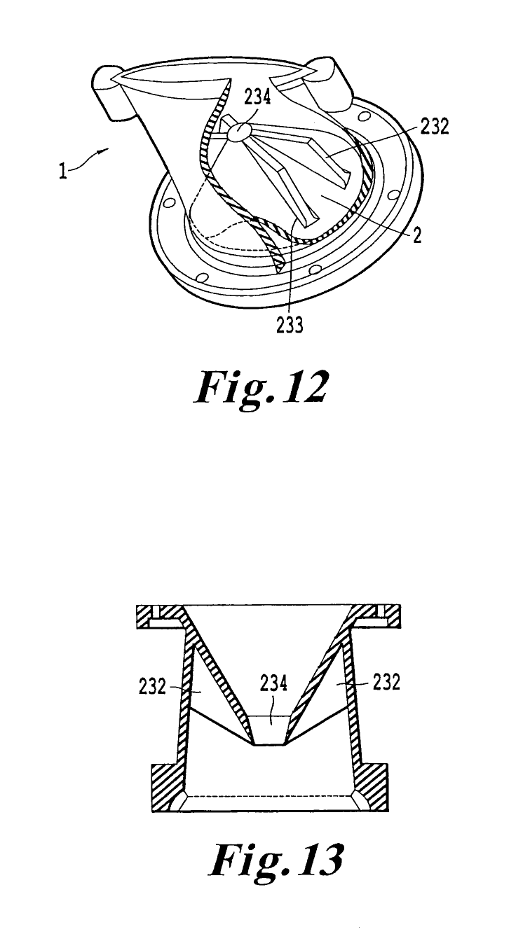

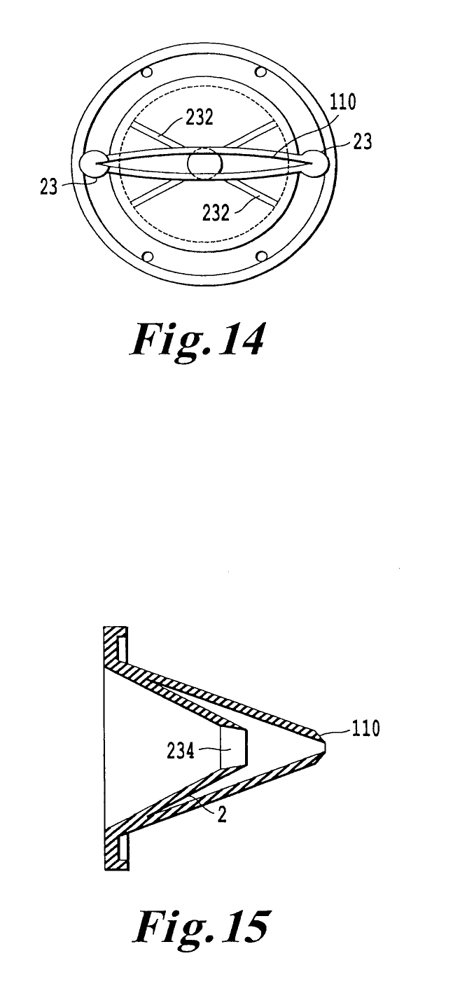

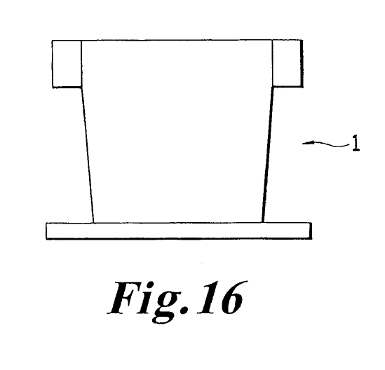

FIGS. 12-16 illustrate an additional embodiment of the invention which is similar to that described above but include ribs 232 provided on the exterior surface portion of the cone 2. These ribs 232 are therefore positioned between the cone and the conoid 1 and have a base 233. In addition, the conoid 1 is shown as including beveled outside edges 110. The internal cone 2 is also provided with a cylindrically shaped channel or opening 234 having a longitudinal length of at least two times the thickness of the cone 2. This channel or opening 234 thus provides an additional length of contact surface for an air tightly contacting an object that is inserted through the cone and thus provides an even more effective seal than that provided in the first embodiment of the present invention. The beveled outside edges 110 are acute angled (for example, at an angle of 30° with respect to the outside surface portion of the conoid 1), as can be understood from a review of FIG. 15. The illustrated embodiment shows four ribs 232 provided but are greater or lesser number of ribs can be provided depending upon the composition and diameter characteristics of the material forming the cone and ribs.

As would be understandable to one of ordinary skill in the art, obviously, numerous modifications and variations of the present invention are possible in light of the above teachings. For example, the seal can be utilized in any structural assembly that permits passage of a member through a seal. This could include, for example, the nozzle of a filling station pump or other type of insertable member that passes through the seal mounted in the passageway of the vehicle that leads to the fuel tank of the vehicle. Other possibilities are clearly possible which would be within the knowledge of one of ordinary skill in the art of providing seals for passageways. It is therefore to be understood that within the scope of the pending claims, the invention may be practiced otherwise than as specifically described therein.

1-21. (canceled)

22. A one-piece trocar seal for a trocar, comprising:
a conoid element having an opening at opposite ends thereof for being positioned in the trocar;
a seal positioned coaxially within said conoid element wherein said seal has an opening at opposite ends thereof; and
a container removably positionable within the trocar and within which the conoid element and seal are positioned.

23. The one-piece seal according to claim 22, wherein said seal is unitarily connected to said conoid element and said container comprises a cylindrical container.

24. The one-piece seal according to claim 22, wherein said rim of the conoid element has a substantially cylindrical shape and is unitarily connected to said seal.

25. The one-piece seal according to claim 22, wherein the conoid element has a chamfered edge at one of said opposite ends thereof and wherein a lubricant is positioned within said seal.

26. The one-piece seal according to claim 22, wherein the seal includes at least one relief groove formed therein.

27. The one-piece seal according to claim 22, which comprises at least one rib member connected to an outer surface of said seal wherein the conoid seal and seal form a one piece assembly.

28. The one-piece seal according to claim 22, wherein said rim of said conoid element and said rim of said seal comprises a single rim member attached to at least one of the ends of said conoid element and said seal.

29. A one-piece seal as claimed in claim 22, which comprises at least one rib member connected to an outer surface of said seal.

30. A one-piece seal according to claim 22, wherein said seal comprises a channel shaped opening of a length which is at least two times a thickness dimension of said seal.
